(19) United States
(12) Patent Application Publication
(10) Pub. No.: US 2023/0187063 A1
(43) Pub. Date: Jun. 15, 2023

(54) SYSTEM AND METHOD FOR ASYNCHRONOUS COMMUNICATION OF INFUSION INFORMATION AND OBTAINING REMOTE ASSISTANCE FOR AN ONGOING INFUSION

(71) Applicant: CareFusion 303, Inc., San Diego, CA (US)

(72) Inventors: Michael WORKMAN, Carlsbad, CA (US); Evan CHEN, San Diego, CA (US); Igor NESTERENKO, San Diego, CA (US); Lisa DIGGETT, Olathe, KS (US); Robert REGEDANZ, San Diego, CA (US)

(21) Appl. No.: 17/923,209

(22) PCT Filed: May 3, 2021
(86) PCT No.: PCT/US2021/030510

§ 371 Date: Nov. 3, 2022

Related U.S. Application Data
(60) Provisional application No. 63/019,842, filed on May 4, 2020.

(51) Int. Cl. G16H 40/67; A61B 5/00

(52) U.S. Cl. CPC G16H 40/67; A61B 5/0002; G16H 20/17

(57) ABSTRACT

A medical device is configured to store, in a session memory, data regarding operational parameters of the medical device, changes to the parameters, and physiological data of a patient associated with the medical device. A clinician may select, via a user interface of the medical device, a portion of the session memory to a remote device with a request for assistance regarding a problem occurring at the medical device. The portion of the session may be reviewed at the remote device in a graphical copy of a user interface of the medical device as it appeared when the data was recorded, and a workflow is generated at the remote device and sent to the medical device to assist the clinician in resolving issues at the medical device. A session may be started without an identification of the patient or a medical component, and the identification updated at a later time.

### TECHNICAL FIELD

The present disclosure is generally related to an electronic or electromechanical medical device configured for medication delivery, and which is configured to communicate with a remote devices.

### BACKGROUND

Patient care units may include modular infusion platforms that are expandable with multiple medication delivery modules to handle more than one type of medication delivery to a patient. Different patients require different levels of treatment, and different parameters for different devices. Each individual infusion device may operate differently, and include a different type of user interface, resulting in a wide variety of display types and parameter variations that may confuse or distract clinicians during infusion and other medication delivery procedures. Failure of a medical device or a medication increases the health risk to the patients of a healthcare facility. Further, assistance to clinicians in resolving issues related to infusion devices may be difficult to obtain, particularly in a busy hospital environment or in urgent medical situations.

### SUMMARY

According to various aspects, the subject technology provides a system and method for communicating infusion information between a medical device and a remote device, and obtaining remote assistance for an ongoing infusion. According to various aspects, the system includes an infusion device having a non-transitory memory and configured to receive and record, in the memory, first physiological data associated with a patient, and a plurality of user modifications to one or more operating parameters of the infusion device, and computer-readable instructions configured to, when executed by one or more processors, cause the one or more processors to perform operations related to communicating session information of the infusion device. In this regard, the operations include receiving, from the infusion device, a user selected portion of an infusion session, the user selected portion of the infusion session being defined by a start time and an end time selected at the infusion device, wherein the infusion session is stored in a memory of the infusion device responsive to an administration of a medication to the patient by the infusion device, receiving, from the infusion device, the portion of the infusion session corresponding to the start time and the end time, the portion of the infusion session including a subset of the first physiological data and a subset of the plurality of modifications received at the infusion device, displaying a graphical representation of the infusion session, the graphical representation including a graphical visualization of the subset of the first physiological data and the subset of the plurality of modifications received at the infusion device, receiving an operating parameter provided to the graphical visualization, the operating parameter corresponding to at least one of the subset of the plurality of modifications, and providing the operating parameter to the infusion device, wherein the operating parameter is displayed by a display screen of the infusion device and the administration of the medication to the patient is adjusted based on the operating parameter.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIG. 2 illustrates an example patient care unit, including a control unit and connected medication delivery modules, according to various aspects of the subject technology.

FIG. 3 depicts an example process for communicating infusion information and obtaining remote assistance for an ongoing infusion, according to aspects of the subject technology.

FIG. 4 is a conceptual diagram illustrating an example electronic system for communicating infusion information and obtaining remote assistance for an ongoing infusion, according to aspects of the subject technology.

FIG. 5 shows a portion of an example user interface for capturing and sharing infusion session information according to aspects of the subject technology.

FIG. 6 shows a portion of an example user interface for sharing infusion session information according to aspects of the subject technology.

FIG. 7 shows a portion of an example user interface for reviewing or sharing infusion session information according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The subject technology provides a system for immediate communication between a clinician in the field and a pharmacy or technician or other clinician. The system includes a electronic or electromechanical medical device configured for medication delivery, and which is configured to communicate with a remote device to receive real time assistance with an ongoing infusion from a remote user. “Real time assistance” generally refers to assistance provided is sufficient time to maintain continuity of a process such as programming a medical device. The clinician may be having a problem setting certain parameters of a medical device, or may be attempting to set parameters to alleviate a medical condition or undesirable response to a current administration of a medication. The clinician may, using a user interface displayed on the medical device, initiate communication with the remote user (or system), who may be more knowledgeable regarding adjustments to the medical device to provide a desirable outcome.

According to various aspects, the medical device is configured to store, in a session memory, data regarding operational parameters of a medical device, changes to the parameters, and physiological data of a patient associated with the medical device. The clinician may select, via the user interface of the medical device, a portion of the session memory to send to a remote device, together with a request for assistance regarding a problem occurring at the medical device. The selected portion of the session may be selected by way of a bookmarking mechanism in the user interface. The selected portion of the session is sent over a network and displayed at the remote device in a graphical copy of the user interface displayed by the medical device, as it appeared when the data was recorded. In other words, the subject technology provides a mechanism by which a clinician at the medical device may bookmark or generate a live snapshot of a selected portion of a session, and send that portion to a remote user (e.g., a pharmacy technician or senior clinician) for playback by the remote user. A workflow may then be generated by the remote user at the remote device and sent to the medical device to assist the clinician in resolving issues at the medical device.

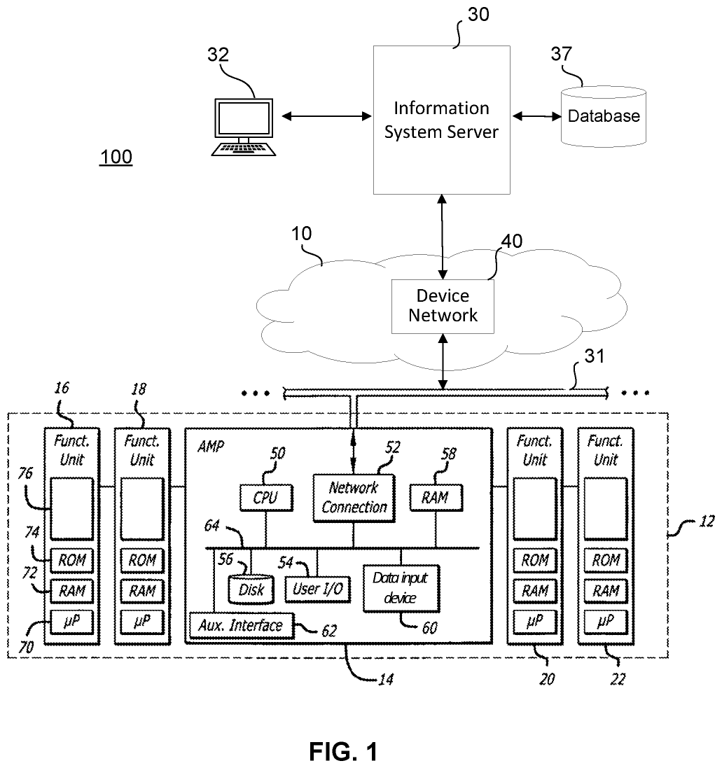

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 40 by which patient care devices 12 (and other devices) communicate in accordance with normal operations.

Additionally, institutional patient care system 100 may incorporate a separate information system server 30, the function of which will be described in more detail below. Moreover, although the information system server 30 is shown as a separate server, the functions and programming of the information system server 30 may be incorporated into another computer, if such is desired by engineers designing the institution's information system. Institutional patient care system 100 may further include one or multiple device terminals 32 for connecting and communicating with information system server 30. Device terminals 32 may include personal computers, personal data assistances, mobile devices such as laptops, tablet computers, augmented reality devices, or smartphones, configured with software for communications with information system server 30 via network 10.

Patient care device 12 comprises a system for providing patient care, such as that described in U.S. Pat. No. 5,713,856 to Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 16, 18, 20, 22. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1 to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 16, 18, 20 and 22, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 16, 18, 20, 22 are any devices for providing care to a patient or for monitoring patient condition. As shown in FIG. 1, at least one of functional modules 16, 18, 20, 22 may be an infusion pump module such as an intravenous infusion pump for delivering medication or other fluid to a patient. For the purposes of this discussion, functional module 16 is an infusion pump module. Each of functional modules 18, 20, 22 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor or an intracranial pressure monitor or the like. Functional module 18, 20 and/or 22 may be a printer, scanner, bar code reader or any other peripheral input, output or input/output device.

Each functional module 16, 18, 20, 22 communicates directly or indirectly with interface unit 14, with interface unit 14 providing overall monitoring and control of device 12. Functional modules 16, 18, 20, 22 may be connected physically and electronically in serial fashion to one or both ends of interface unit 14 as shown in FIG. 1, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 16, 18, 20, 22 may include module-specific components 76, a microprocessor 70, a volatile memory 72 and a nonvolatile memory 74 for storing information. It should be noted that while four functional modules are shown in FIG. 1, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 16.

While each functional module may be capable of a least some level of independent operation, interface unit 14 monitors and controls overall operation of device 12. For example, as will be described in more detail below, interface unit 14 provides programming instructions to the functional modules 16, 18, 20, 22 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in HIS server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication

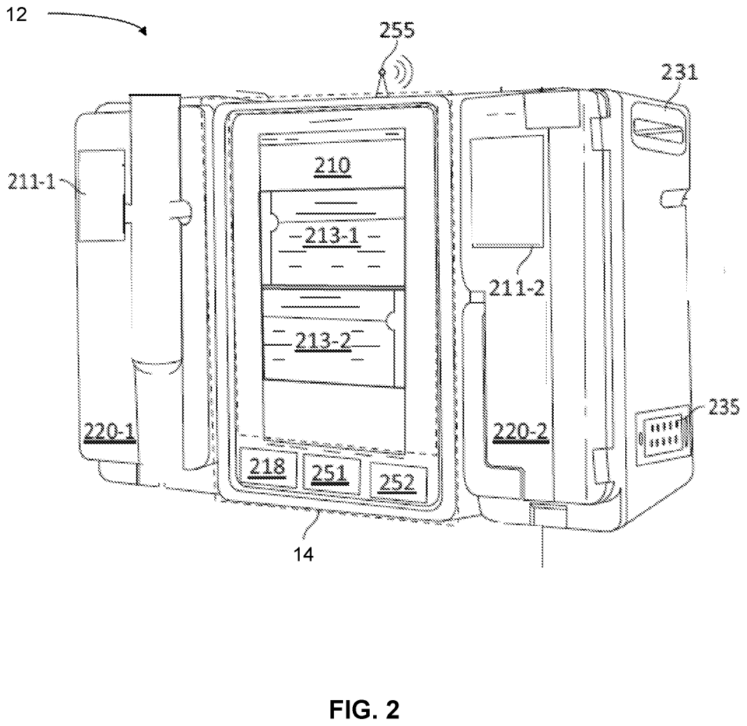

FIG. 2 illustrates an example PCU 12, including a control module 14 together with connected medication delivery modules 220-1 and 220-2 (e.g., modules 16, 18, 20, 22), according to various aspects of the subject technology. In some embodiments, medication delivery modules 220 include plug-in ports 231 and 235 for expansion. Accordingly, a new medication delivery module may be attached to PCU 12 by coupling a connector through plug-in ports 231 and 235. Plug-in port 235 may include electrical terminals so that the added medication delivery module 220 may transmit and receive information to and from a control module 14. In some embodiments, the added medication delivery module 220 may also receive power from control module 14 through the plug-in port 235. Control module 14 may include a main display 210, a memory 251 and a processor 252 (e.g., memory 151 and processor 152). Module displays 211-1 and 211-2 (hereinafter, collectively referred to as “module displays 211”) may be configured to display operational parameters and medication delivery status, and further information associated with each of medication delivery modules 220. According to various implementations, module displays 211 may also display physiological data (e.g., vital signs) associated with a patient.

Main display 210 is configured to display one or more user interfaces 213 for the display of operational parameters or other data associated with a module 220, and/or physiological parameters associated with the patient. Main display 210 may include multiple user interfaces 213, with each individual user interface graphically displaying information for a respective one of medication modules 220, including information also displayed on a corresponding module display 211. In some embodiments, control module 14 includes a communications module 218 and an antenna 255, configured to communicate wirelessly with a controller (e.g., controller 110), or with a network.

With reference to FIGS. 1 and 2, when a medication delivery module 220 initiates an infusion of a medication to the patient, the control module 14 is configured to create and manage an infusion session within a memory of the control module (or related module). For the purpose of this disclosure, the infusion session includes state information of the PCU 12, its control module 14, and/or its associated modules, which is recorded and saved to memory during a particular period of time. The state information includes, but is not limited to, records of parameter values received or utilized by the PCU, its control module, and/or its associated modules during the period of time, records of physiological data collected during the period of time, and/or records of operational characteristics of the PCU, its control module, or its associated modules during the period of time (e.g., power level, software version, firmware version, logged in user, infusion set(s) coupled with the device). During the infusion, physiological data associated with the patient is recorded within the session, operating parameter values, and any modifications to the operating parameters of the PCU, its control module, and/or modules are also recorded in the session. The session may include information identifying a value entered, but not used to administer a fluid, and later changed. The session may include information identifying an error for a value entered such as a value that does not correspond to safety criteria.

If not already logged into the PCU 12, the clinician may scan his or her badge proximate to a sensor on the PCU (e.g., part of communication module 218), and the PCU may attempt to authenticate the clinician by sending the clinician's scanned identification to server 30. The clinician's badge may incorporate a radio frequency identification device (RFID), which is read by a scanner integrated with the PCU, or a portable scanner associated with the PCU. The clinician may scan his or her badge at the control module 14 to identify and authorize the clinician to initiate the administration of a medication. Once the clinician is associated with the PCU and/or module(s), the clinician's identification is associated with the session. The same is applicable with a patient. The clinician may scan the patient's wristband with a portable scanner, or using the sensor on the PCU 14 (or its control module) to associate the patient with the PCU and/or module(s) (and a session).

During the infusion, the clinician may experience a problem with setting a parameter, not know the correct value to set based on the current state of the patient, or the patient may have an undesirable reaction to the current infusion or may become at risk due to a deteriorating health condition evidenced by physiological readings. The clinician may, using an input interface of the control module 21, remotely contact a remote user to receive assistance with the issue in real time. The control module 14 is configured to initiate communication with a remote device 32 over network 40, by which the clinician and the remote user may communicate in real time (e.g., synchronously without substantial perceivable delay) through the PCU.

Once a communication session is open between the PCU and the remote device 32, the remote device 32 may receive a real time representation of the PCU's display screen 210, and the user and the clinician may communicate by way of audio or video using audio and/or video devices (e.g., a microphone and/or camera) incorporated in the PCU and remote devices, respectively. In this manner, the remote user may review parameters of the control module 14 and any module(s) associated with the control module in real time (e.g., synchronously without substantial perceivable delay or within sufficient time to allow the control module to continue processing without significant clinical delay). In some implementations, the control module 14 may automatically push keystrokes input at the control module and/or module(s) to the remote device 32 for review by the remote user. The remote user may also receive a history of actions performed at the pump and/or control module as part of the push.

According to various implementations, the clinician may select, or bookmark, a particular period of time for which to record data collected by the control module 14, or of previously recorded data. In this regard, user interface 213 may include a menu for selecting a certain start an end time for the data recording. The user interface 213 may also include an icon to start and/or stop recording. The selection may be for a particular period of time within a current infusion session, or may be selected to initiate an infusion session for the selected period of time.

The user interface 213 may also enable the clinician to send a selected portion of the infusion session to the remote device (e.g., defined by the start and end times), for review by the remote user. Additionally or in the alternative, once the PCU is connected to the remote device, the remote deice 32 may display a user interface that enables the remote user to request or pull the infusion session to the remote device from the PCU, or other cause the PCU to transmit the infusion session or portion thereof to the remote device. The PCU may then send, over the network 40, the selected portion of the infusion session, including at least a subset of physiological data obtained from sensors monitoring the patient, current values of operating parameters associated with the current infusion, and an indication of any modifications received at the infusion device.

Remote device 32 may display a copy of the user interfaces displayed on the display screen 210, or other wise generate a graphical representation of the infusion session and associated data transmitted to the remote device from the PCU, providing an end-to-end visualization of an infusion. Remote device 32 may replay operational data modifications and patient physiological data as it appeared within the user interface 213 of the PCU between the start time and the end time, including physiological data and modifications as they were made at the PCU or appeared within the user interface 213 when received at the PCU. Remote device 32 may also display a menu that allows the remote user to select parameters for display that may not be displayed on the display screen. For example, the remote user may be able to access all underlying operating parameters of an ongoing infusion, the infusion device, or control module, whether or not those parameters are available for display to the clinician at the PCU.

The remote user may then use the displayed user interface at the remote device 32 to remotely adjust operating parameters of the PCU (which were, e.g., part of the session). In this regard, the remote user may adjust an operating parameter, and the remote device may send the adjusted operating parameter to the PCU. The adjusted operating parameter may then be displayed by display screen 210 of the PCU, or otherwise automatically updated at the PCU, and the administration of the medication to the patient medication may be adjusted based on the adjusted operating parameter.

In some implementations, the remote device 32 may be configured to identify an alert condition indicative of an undesirable physiological response, based on a subset of the received physiological data and/or a subset of the parameters or modifications made at the infusion device. This may be accomplished by comparing received physiological data and/or current device operating parameters with physiological measurements and/or operating parameters (or ranges thereof) known for physiological conditions (which may be stored in database 37). The remote device 32 may then generate a workflow for resolving the alert condition, including further modifications to operational parameters of the PCU (or infusion device administering the medication). The workflow may be sent from the remote device 32 to the control unit of the PCU for display at the PCU. The display screen 210 may then display a graphical representation of the workflow for review by the clinician. The clinician may then adjust or enter operational parameters according to the workflow, resulting in an adjustment or correction to the administration of the medication. The PCU may then detect whether the alert condition was resolved and report the condition was resolved to the remote device 32.

The remote communication between the PCU and the remote device may also be used for secondary authorization purposes. For example, the clinician logged in to the PCU may not have authorization to administer a particular narcotic or chemotherapy drug. User interface 213 may include one or more controls for contacting an authorized clinician to approve the administration, remotely. Database 37 may include associations between medication orders, patients, and/or clinicians, and may include information regarding when a medication was dispensed, wherefrom, and who may authorize the use of such medication for which patients and in what care areas. Database 37 may also include information on how to contact an authorized remote user. For example, remote device 32 may be a mobile device, and the database may include connection information for connecting to a client application and user interface on the mobile device.

When a restricted medication is installed in a drug infusion module, the PCU may identify the drug as being restricted (e.g., based on an RFID scan of an RFID chip on the medicine container or bag) and lock the infusion module (preventing delivery of the drug) until a secondary authorization can be obtained. User interface 213 may then provide a list of authorized clinicians, and provide a control for contacting the authorized clinicians through the interface. In some implementations, display screen 210 or user interface 213 may automatically set to standby mode pending a remote authorization to proceed from an authorized remote user. Such mechanism may also be useful in clinician training. The secondary authorization may be by the remote user via the remote device 32.

According to various implementations, the infusion session managed by the PCU and/or the corresponding module(s) includes a data structure which enables retroactive analysis of a particular patient's infusion session even when that patient is not known until much later during the session. When initiated, the infusion session begins saving data with identification information on-hand. If a patient or medication component is unknown, the PCU assigns a pseudo identifier for the patient or medication. Such identifier is used as a placeholder so that a complete picture of the infusion session may be compiled at a later time. For example, an infusion may be started for an unknown patient using an unknown IV set. The PCU will start an infusion session with a first pseudo identifier for the patient and a second pseudo identifier for the IV set. When the patient is later scanned, the PCU will associate the entire session (back to when it was initiated) with the proper patient identifier. The IV set may be later scanned and also associated with the entire session. In some implementations, user interface 213 may request conformation before the association is made to the entire session, or may provide the clinician with options for selecting a portion (e.g., time period) of the session to associate the identifier.

When the infusion session is started, the PCU may assign a pseudo identifier representative of a patient, and pseudo identifiers for any disposable components (e.g., IV set) used in the infusion, and pseudo identifiers for the medication being administered. If this information was previously scanned by the clinician when the infusion is set up then the known identifiers may be used. However, by using pseudo identifiers, having all of the information up front is no longer necessary. Over the course of an infusion, the clinician may make one or more modifications to operating parameters, for example, to adjust the administration of the medication. The PCU may also receive and record in the infusion session, physiological data associated with the patient. All of the collected data, and any modifications to the data or operating parameters, are stored in connection with the infusion session, irrespective of whether pseudo identifiers or known identifiers are being used by the infusion session.

Later, after an amount of medication is administered to the patient and/or medication adjustments are made, the clinician may input a known identifier of a known patient, disposable, or medication, for association with the current administration of the medication. The pump will then assign the known identifier to the infusion session in substitution of the pseudo identifier used for the unknown patient, type of component, or unknown medication. According to some implementations, the pseudo identifier may be associated after the session has ended. For example, user interface 213 may include an option for selection of past infusion sessions, and the clinician, on selecting a past infusion session, may be presented with a list of unknown identifiers currently associated with the session. The clinician may then scan or enter a known identifier to associate with the session.

Creation of the infusion session creates a place holder for the session subject, for example, a patient. This allows the subject technology, using the place holder data, to back associate previous events that occurred before patient ID (or other ID) become known. For example, infusion records on a previously unknown patient can be linked back to a named or otherwise identified patient. The session facilitates the ability to filter to a span of relevant events that happen within its bounds, or ‘bookends’. Additionally, each session is assigned a session ID. While the pump ID may be known and assigned to the session, the patient ID and other IDs for the medication or various components may be unknown when the session ID is created, and can be assigned later.

The control unit of PCU is configured to generate a graphical representation of the infusion session, and display (e.g., in user interface 213) the graphical representation, including a graphical visualization of all parameters of the infusion during the session and any modifications any modifications to the parameters, together with physiological data obtained during the session. The graphical representation may include pseudo identifiers for unknown data until such data is substituted with known identifiers. At that time, the graphical representation is displayed with the known patient identifiers. Additional data (e.g., operating parameters and modifications thereto, physiological data, and the like) received after the known patient identifier (or component identifier) is assigned to the infusion session and will be displayed together data points received before the known identifier was assigned to the session. The additional data may include modifications received from the remote device 32, as described previously.

Computer program code for carrying out operations of the subject technology may be written in an object oriented programming language such as, for example, JAVA®, Smalltalk, or C++. However, the computer program code for carrying out operations of the subject technology may also be written in conventional procedural programming languages, such as the “C” programming language, in an interpreted scripting language, such as Perl, or in a functional (or fourth generation) programming language such as Lisp, SML, Forth, or the like. The software may also be written to be compatible with HLA-7 requirements.

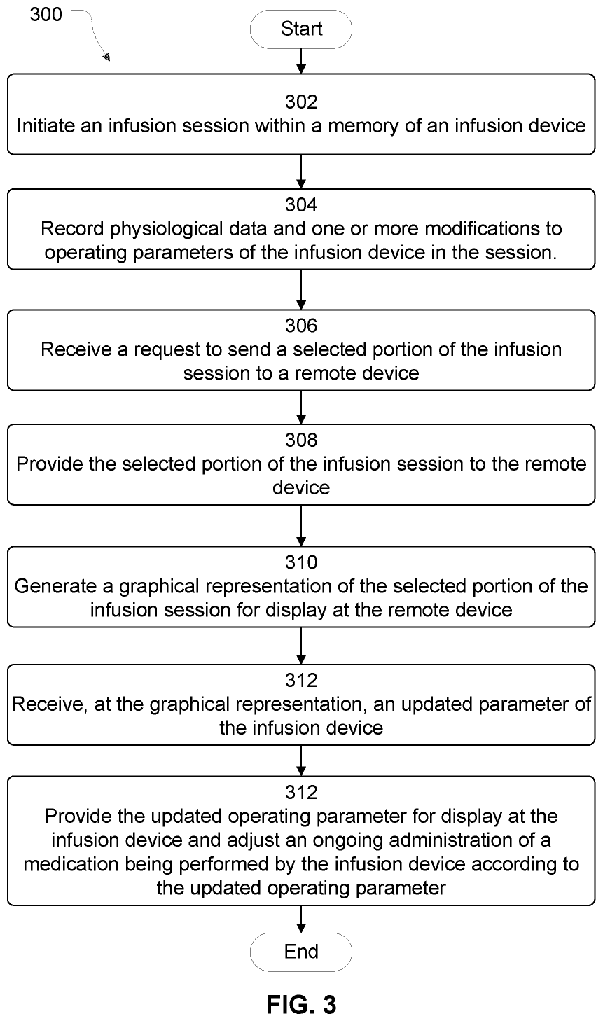

FIG. 3 depicts an example process for communicating infusion information and obtaining remote assistance for an ongoing infusion, according to aspects of the subject technology. For explanatory purposes, the various blocks of example process 300 are described herein with reference to FIGS. 1 and 2, and the components and/or processes described herein. The one or more of the blocks of process 300 may be implemented, for example, by one or more computing devices including, for example, PCU 12, or component thereof. In some implementations, one or more of the blocks may be implemented apart from other blocks, and by one or more different processors or devices. Further for explanatory purposes, the blocks of example process 300 are described as occurring in serial, or linearly. However, multiple blocks of example process 300 may occur in parallel. In addition, the blocks of example process 300 need not be performed in the order shown and/or one or more of the blocks of example process 300 need not be performed.

In the depicted example, an infusion session is initiated within a memory of an infusion device (302). For the purpose of this example, the infusion device may include the PCU 12, control unit 14, or an associated module. The infusion session may be created responsive to the initiating of an administration of a medication to a patient by the infusion device. Physiological data associated with the patient, and one or more modifications to one or more operating parameters of the infusion device, are then recorded in the infusion session (304) during the medication administration.

The infusion device receives a request to send a selected portion of the infusion session to a remote device (306). The selected portion of the infusion session may be defined by a start time and an end time selected at a user interface 213 of the infusion device.

The selected portion of the infusion session is then provided to the remote device (308). The infusion session may be transmitted from the infusion device over network 40 to a remote device 32. In some implementations, the transmission may occur directly, peer-to-peer. In some implementations, the transmission may be facilitated by server 30. For example, server 30 may collect all data and provide a web-based interface for display of data to all connected devices. The portion provided to the remote device 32 may correspond to the start time and the end time selected by a clinician at user interface 213. The portion of the infusion session may include a subset of the physiological data and modifications received at the infusion device.

A graphical representation of the transmitted portion of the infusion session is generated for display at the remote device 32 (310). The graphical representation may include a graphical visualization the transmitted subset of physiological data and modifications received at the infusion device. A remote user may then review the data and provide feedback to the clinician at the infusion device using audio, textual, messaging, or video communication in real time (e.g., synchronously without substantial perceivable delay).

In the depicted example, an updated operating parameter is provided to the graphical visualization at the remote device (e.g., by a remote user) and sent to the infusion device (312) by the application running on the remote device. The operating parameter may correspond to at least one of the operating parameters or modifications received in the infusion session. The updated operating parameter is then provided for display in the user interface 213 on the display screen 210 of the infusion device, and the ongoing administration of the medication to the patient is automatically adjusted by the infusion device based on the received updated operating parameter (314).

Many of the above-described example 300, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

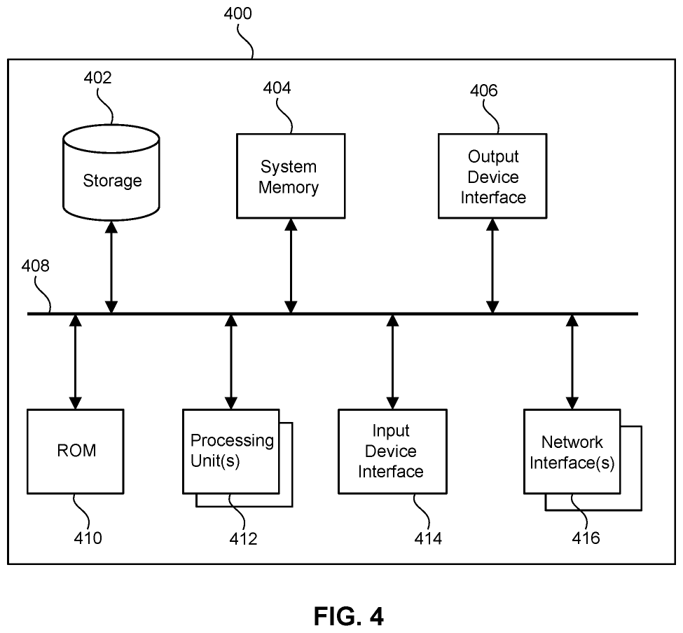

FIG. 4 is a conceptual diagram illustrating an example electronic system 400 for communicating infusion information and obtaining remote assistance for an ongoing infusion, according to aspects of the subject technology. Electronic system 400 may be a computing device for execution of software associated with one or more portions or steps of process 400, or components and processes provided by FIGS. 1-3, including but not limited to information system server 30, production server 204, computing hardware within patient care device 12, or remote device 32. Electronic system 400 may be representative, in combination with the disclosure regarding FIGS. 1-3. In this regard, electronic system 400 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 400 may include various types of computer readable media and interfaces for various other types of computer readable media. In the depicted example, electronic system 400 includes a bus 408, processing unit(s) 412, a system memory 404, a read-only memory (ROM) 410, a permanent storage device 402, an input device interface 614, an output device interface 406, and one or more network interfaces 416. In some implementations, electronic system 400 may include or be integrated with other computing devices or circuitry for operation of the various components and processes previously described.

Bus 408 collectively represents all system, peripheral, and chipset buses that communicatively connect the numerous internal devices of electronic system 400. For instance, bus 408 communicatively connects processing unit(s) 412 with ROM 410, system memory 404, and permanent storage device 402.

From these various memory units, processing unit(s) 412 retrieves instructions to execute and data to process in order to execute the processes of the subject disclosure. The processing unit(s) can be a single processor or a multi-core processor in different implementations.

ROM 410 stores static data and instructions that are needed by processing unit(s) 412 and other modules of the electronic system. Permanent storage device 402, on the other hand, is a read-and-write memory device. This device is a non-volatile memory unit that stores instructions and data even when electronic system 400 is off. Some implementations of the subject disclosure use a mass-storage device (such as a magnetic or optical disk and its corresponding disk drive) as permanent storage device 402.

Other implementations use a removable storage device (such as a floppy disk, flash drive, and its corresponding disk drive) as permanent storage device 402. Like permanent storage device 402, system memory 404 is a read-and-write memory device. However, unlike storage device 402, system memory 404 is a volatile read-and-write memory, such a random access memory. System memory 404 stores some of the instructions and data that the processor needs at runtime. In some implementations, the processes of the subject disclosure are stored in system memory 404, permanent storage device 402, and/or ROM 410. From these various memory units, processing unit(s) 412 retrieves instructions to execute and data to process in order to execute the processes of some implementations.

Bus 408 also connects to input and output device interfaces 414 and 406. Input device interface 414 enables the user to communicate information and select commands to the electronic system. Input devices used with input device interface 414 include, e.g., alphanumeric keyboards and pointing devices (also called “cursor control devices”). Output device interfaces 406 enables, e.g., the display of images generated by the electronic system 400. Output devices used with output device interface 406 include, e.g., printers and display devices, such as cathode ray tubes (CRT) or liquid crystal displays (LCD). Some implementations include devices such as a touchscreen that functions as both input and output devices.

Also, as shown in FIG. 4, bus 408 also couples electronic system 400 to a network (not shown) through network interfaces 416. Network interfaces 416 may include, e.g., a wireless access point (e.g., Bluetooth or WiFi) or radio circuitry for connecting to a wireless access point. Network interfaces 416 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 400 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

To provide output to a user or via a display device, output controllers may be included. Output controllers may include controllers for any of a variety of display devices for presenting information to a user, whether a human or a machine, whether local or remote. If one of the display devices provides visual information, this information typically may be logically and/or physically organized as an array of picture elements. A user interface controller may include software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML, or XML, documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT®, Windows XP, Windows 7, Windows 8, Windows 10, iOS, Android, Sun Solaris, Linux, OS/400, Compaq Tru64 Unix, SGI IRIX, Siemens Reliant Unix, real-time operating systems such as FreeRTOS, and others.

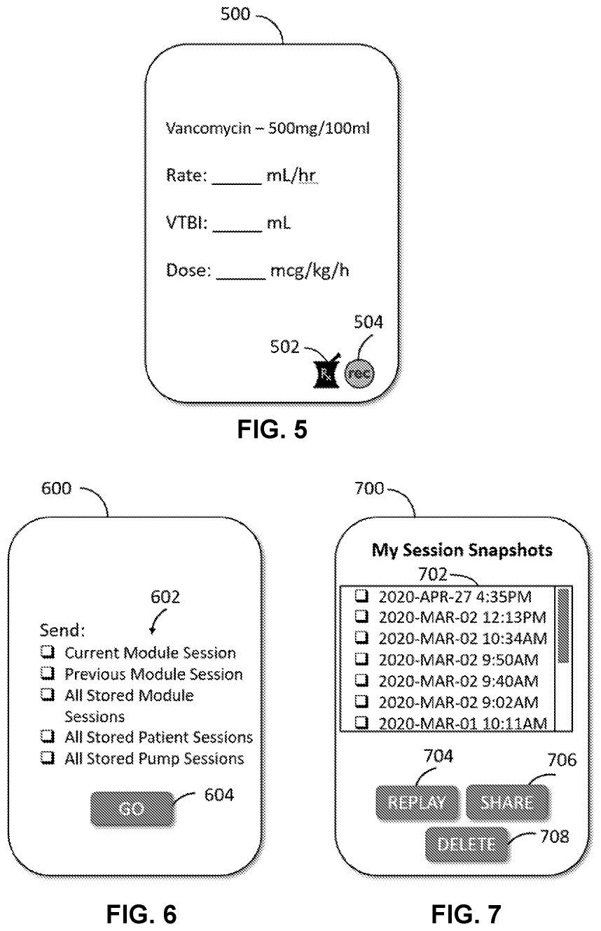

FIG. 5 shows a portion of an example user interface for capturing and sharing infusion session information according to aspects of the subject technology. The interface 500 may be presented via one or more of the devices described. The interface 500 is an example of an interface that the patient care device may present to collect information for infusing a fluid to a patient. In the example shown, the fluid to be infused is vancomycin. As patient care device receives values for the infusion parameters, the values may be compared to corresponding safety limits. If the entered value does not correspond with the safety limit, the interface 500 may display and/or activate a help control element 502. The help control element 502, when interacted with by a user (e.g., touched, pressed, selected), may initiate a request for help such as from a pharmacist who can assist in identifying and troubleshooting a programming error. Initiating the help may include presenting a second interface such as shown in FIG. 6.

FIG. 6 shows a portion of an example user interface for sharing infusion session information according to aspects of the subject technology. The interface 600 may be presented upon activation of a help control element to receive one or more inputs identifying what session information to share. In FIG. 6, the interface 600 includes a sharing control element 602. The sharing control element 602 includes several selectable options to identify the session information to share as part of the help request. The available options may be configured for all users of the system, based on user attribute such as role (e.g., traveling nurse, manager, supervisor, physician), based on device location (e.g., care area), or other parameter available to the patient care device.

The interface 600 may include a control element 604 to transmit the selected session information to the appropriate assistance device. The assistance device may order received requests based on the information included in the session information such as drug, drug type, pump location (e.g., care area), clinician requesting assistance, infusion status (e.g., running infusion or new infusion), or other parameter detectable by the assistance device. In some implementations, the control element 604 may be deactivated until at least one selection is made from the sharing control element 602. Deactivation may include hiding the control element 604 from the display or ignoring interactions with the control element 604. The interface 600 may include additional or alternative control elements to receive inputs for the help session. Examples of such elements include text entry control elements, voice recording control elements, video recording control elements, or the like.

Returning to FIG. 5, the interface 500 may include a record control element 504. The record control element 504, when activated, may cause the patient care device to store a unique annotation for the current session. The unique annotation allows a user to return to the specific pump state at the time of annotation. Such annotations allow a user to share best practices by capturing specific programming sessions, capture errors or discrepancies for the patient care device or supporting configurations, or collect evidence of a particular fluid administration. The annotations may be associated with the user who logged into the patient care device.

FIG. 7 shows a portion of an example user interface for reviewing or sharing infusion session information according to aspects of the subject technology. The interface 700 may be used to review sessions recorded by a user. In some implementations, a user may access snapshot from any patient care device within the user's clinical network. The interface 700 may be available based on patient association. For example, the interface 700 may not be accessible when the patient care device is associated with a patient. This can protect the privacy of the session data and may further enhance clinical safety by avoiding confusion between replayed programs and the actual running program.

The interface 700 includes a snapshot selector element 702. The snapshot selector element 702 receives a selection of one or more snapshots to view, share, or delete. Action elements may be provided to adjust operation of the patient care device based on the selected snapshots. As shown, the interface 700 includes a replay control element 704 which may initiate replay of the selected snapshot. The replay may include configuring the interface of the patient care device to display the programming steps captured in the snapshot. In some implementations, the replay may include adjusting or presenting information via corresponding modules attached to the patient care device.

As shown, the interface 700 includes a share control element 706 which allows a user to transmit a snapshot to another authorized user. The authorization may be based on user permissions, roles, organizational hierarchy or relationship between users, care area, or other criteria detectable by or via the system. Sharing session information may include transmitting a message including an identifier for referencing the session rather than individual data elements included in the session. Using the identifier, an authorized, receiving party can query the system for the data elements included in the session. In some implementations, a receiving party may be authorized to receive partial information for the identified session. For example, the drug information and programmed parameters may be accessible but patient information may be masked, anonymized, or otherwise suppressed from presentation to the receiving party. The interface 700 may include an option to share a snapshot with a support entity such as the manufacturer of the device. These shares may, in some instances, be characterized as customer feedback or assistance requests. The interface 700 may include additional or alternative control elements to receive inputs for the session snapshots or share requests. Examples of such elements include text entry control elements, voice recording control elements, video recording control elements, or the like.

As shown in FIG. 7, the interface includes a delete control element 706. Deleting a selected snapshot may cause the system to remove the user's annotation for the snapshot along with any additional information provided by the user and associated with the snapshot such as voice recordings, text annotations, video recordings, or the like. Deleting a snapshot does not delete the underlying session information which may be reviewed later and, in some instances, associated with a new annotation by the same or a different user.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device, diagnostic device, monitoring device, or server in communication therewith.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some embodiments, a server transmits data (e.g., an HTML, page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The term website, as used herein, may include any aspect of a website, including one or more web pages, one or more servers used to host or store web related content, etc. Accordingly, the term website may be used interchangeably with the terms web page and server. The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A method, comprising:
starting an infusion session within a memory of an infusion device responsive to an administration of a medication to a patient by the infusion device;
recording, in the infusion session, first physiological data associated with the patient, and a plurality of modifications to one or more operating parameters of the infusion device;
receiving, from the infusion device, a request to send a selected portion of the infusion session to a remote device, the selected portion of the infusion session being defined by a start time and an end time selected at the infusion device;
providing, to the remote device over a network, from the infusion device, the portion of the infusion session corresponding to the start time and the end time, the portion of the infusion session including a subset of the first physiological data and a subset of the plurality of modifications received at the infusion device;
generating a graphical representation of the infusion session for display at the remote device, the graphical representation including a graphical visualization of the subset of the first physiological data and the subset of the plurality of modifications received at the infusion device;
receiving, from the remote device, an updated operating parameter provided to the graphical visualization, the updated operating parameter corresponding to at least one of the subset of the plurality of modifications; and
providing the operating parameter to the infusion device, wherein the updated operating parameter is displayed by a display screen of the infusion device and the administration of the medication to the patient is adjusted based on the updated operating parameter.

2. The method of claim 1, further comprising:
identifying, based on the subset of the first physiological data and the subset of the plurality of modifications received at the infusion device, an alert condition indicative of an undesirable physiological response;
generating, at the remote device, a workflow for resolving the alert condition, including further modifications to operational parameters of the infusion device; and
providing a graphical representation of the workflow to the infusion device for display on the display screen of the infusion device.

3. The method of claim 2, further comprising:
displaying, on the display screen, the graphical representation of the workflow;
receiving, from the infusion device, user input to adjust or enter operational parameters according to the workflow;
adjusting the administration of the medication based on the user input; and
determining that the alert condition was resolved.

4. The method of claim 1, further comprising:
graphical visualization comprises a copy of a user interface displayed on the infusion device and a replay of operational data modifications and patient physiological data as it appeared within the user interface between the start time and the end time, including the subset of the first physiological data and the subset of the plurality of modifications as it appeared within the user interface when received at the infusion device.

5. The method of claim 1, further comprising:
assigning to the infusion session, by an infusion device, before an identity of the patient is known, a pseudo identifier representative of the patient;
receiving, by the infusion device, one or more modifications at least one operating parameter of the infusion device, the administration of the medication being adjusted according to the one or more modifications;
receiving, after the medication is adjusted, second physiological data associated with the patient and indicative of a response to the adjusted administration of the medication;
recording, in association with the pseudo identifier, the one or modifications and the physiological data to the infusion session;
receiving, after the medication is adjusted, a known patient identifier of a known patient for association with the administration of the medication; and
assigning the known patient identifier to the infusion session in substitution of the pseudo identifier;
generating a graphical representation of the infusion session, the graphical representation including a graphical visualization of the recorded one or more modifications and second physiological data in association with the known patient identifier, and at least one additional physiological data point received after the known patient identifier is assigned to the infusion session.

6. The method of claim 5, further comprising:
receiving, from the remote device, a further modification of at least one operational parameter of the infusion device; and
modifying, at the infusion device, the at least one operational parameter of the infusion device according to the received further modification to further adjust the administration of the medication to the patient; and
recording the modification of the at least one operational parameter to the infusion session in association with the known patient.

7. The method of claim 1, further comprising:
assigning a pseudo identifier representative of an intravenous (IV) set to the infusion session, by an infusion device, before an identity of the IV set is known;
receiving, by the infusion device, one or more modifications at least one operating parameter of the infusion device, the administration of the medication being adjusted according to the one or more modifications;
receiving, after the medication is adjusted, second physiological data associated with the patient and indicative of a response to the adjusted administration of the medication;
recording, in association with the pseudo identifier, the one or modifications and the physiological data to the infusion session;
receiving, after the medication is adjusted, a known IV set identifier of a known type of IV set for association with the administration of the medication; and
assigning the known IV set identifier to the infusion session in substitution of the pseudo identifier;
generating a graphical representation of the infusion session, the graphical representation including a graphical visualization of the recorded one or more modifications and second physiological data in association with the known IV set identifier, and at least one additional physiological data point received after the known IV set identifier is assigned to the infusion session.

8. A system, comprising:
an infusion device having a non-transitory memory and configured to receive and record, in the memory, first physiological data associated with a patient, and a plurality of user modifications to one or more operating parameters of the infusion device; and
computer-readable instructions configured to, when executed by one or more processors, cause the one or more processors to:
receive, from the infusion device, a user selected portion of an infusion session, the user selected portion of the infusion session being defined by a start time and an end time selected at the infusion device, wherein the infusion session is stored in a memory of the infusion device responsive to an administration of a medication to the patient by the infusion device;
receive, from the infusion device, the portion of the infusion session corresponding to the start time and the end time, the portion of the infusion session including a subset of the first physiological data and a subset of the plurality of modifications received at the infusion device;
display a graphical representation of the infusion session, the graphical representation including a graphical visualization of the subset of the first physiological data and the subset of the plurality of modifications received at the infusion device;
receive an updated operating parameter provided to the graphical visualization, the updated operating parameter corresponding to at least one of the subset of the plurality of modifications; and
provide the updated operating parameter to the infusion device, wherein the operating parameter is displayed by a display screen of the infusion device and the administration of the medication to the patient is adjusted based on the updated operating parameter.

9. The system of claim 8, wherein the computer-readable instructions are further configured to, when executed, cause the one or more processors to:
identify, based on the subset of the first physiological data and the subset of the plurality of modifications received at the infusion device, an alert condition indicative of an undesirable physiological response;
generate a workflow for resolving the alert condition, including further modifications to operational parameters of the infusion device; and
provide a graphical representation of the workflow to the infusion device for display on the display screen of the infusion device.

10. The system of claim 9, wherein the infusion device is configured to:
display, on the display screen, the graphical representation of the workflow;
receive user input to adjust or enter operational parameters according to the workflow;
adjusting the administration of the medication based on the user input; and
determining that the alert condition was resolved.

11. The system of claim 8, wherein the graphical visualization comprises a copy of a user interface displayed on the infusion device and a replay of operational data modifications and patient physiological data as it appeared within the user interface between the start time and the end time, including the subset of the first physiological data and the subset of the plurality of modifications as it appeared within the user interface when received at the infusion device.

12. The system of claim 8, wherein the infusion device is configured to:
assign to the infusion session, by an infusion device, before an identity of the patient is known, a pseudo identifier representative of the patient;
receive, by the infusion device, one or more modifications at least one operating parameter of the infusion device, the administration of the medication being adjusted according to the one or more modifications;
receive, after the medication is adjusted, second physiological data associated with the patient and indicative of a response to the adjusted administration of the medication;
record, in association with the pseudo identifier, the one or modifications and the physiological data to the infusion session;
receive, after the medication is adjusted, a known patient identifier of a known patient for association with the administration of the medication; and
assign the known patient identifier to the infusion session in substitution of the pseudo identifier;
generate a graphical representation of the infusion session, the graphical representation including a graphical visualization of the recorded one or more modifications and second physiological data in association with the known patient identifier, and at least one additional physiological data point received after the known patient identifier is assigned to the infusion session.

13. The system of claim 12, wherein the infusion device is configured to:
receive, from the one or more processors, a further modification of at least one operational parameter of the infusion device; and
modify the at least one operational parameter of the infusion device according to the received further modification to further adjust the administration of the medication to the patient; and
record the modification of the at least one operational parameter to the infusion session in association with the known patient.

14. The system of claim 12, wherein the infusion device is configured to:
assign a pseudo identifier representative of an intravenous (IV) set to the infusion session, by an infusion device, before an identity of the IV set is known;
receive, by the infusion device, one or more modifications at least one operating parameter of the infusion device, the administration of the medication being adjusted according to the one or more modifications;
receive, after the medication is adjusted, second physiological data associated with the patient and indicative of a response to the adjusted administration of the medication;
record, in association with the pseudo identifier, the one or modifications and the physiological data to the infusion session;
receive, after the medication is adjusted, a known IV set identifier of a known type of IV set for association with the administration of the medication; and
assign the known IV set identifier to the infusion session in substitution of the pseudo identifier;
generate a graphical representation of the infusion session, the graphical representation including a graphical visualization of the recorded one or more modifications and second physiological data in association with the known IV set identifier, and at least one additional physiological data point received after the known IV set identifier is assigned to the infusion session.

15. A non-transitory machine-readable storage medium embodying instructions that, when executed by one or more processors, cause the one or more processors to perform operations comprising:
starting an infusion session within a memory of an infusion device responsive to an administration of a medication to a patient by the infusion device;
recording, in the infusion session, first physiological data associated with the patient, and a plurality of modifications to one or more operating parameters of the infusion device;
receiving, from the infusion device, a request to send a selected portion of the infusion session to a remote device, the selected portion of the infusion session being defined by a start time and an end time selected at the infusion device;
providing, to the remote device over a network, from the infusion device, the portion of the infusion session corresponding to the start time and the end time, the portion of the infusion session including a subset of the first physiological data and a subset of the plurality of modifications received at the infusion device;
generating a graphical representation of the infusion session for display at the remote device, the graphical representation including a graphical visualization of the subset of the first physiological data and the subset of the plurality of modifications received at the infusion device;
receiving, from the remote device, an updated operating parameter provided to the graphical visualization, the updated operating parameter corresponding to at least one of the subset of the plurality of modifications;
providing the operating parameter to the infusion device, wherein the updated operating parameter is displayed by a display screen of the infusion device and the administration of the medication to the patient is adjusted based on the updated operating parameter.

16. The non-transitory machine-readable storage medium of claim 15, wherein the operations further comprise:
identifying, based on the subset of the first physiological data and the subset of the plurality of modifications received at the infusion device, an alert condition indicative of an undesirable physiological response;
generating, at the remote device, a workflow for resolving the alert condition, including further modifications to operational parameters of the infusion device; and
providing a graphical representation of the workflow to the infusion device for display on the display screen of the infusion device.

17. The non-transitory machine-readable storage medium of claim 16, wherein the operations further comprise:
displaying, on the display screen, the graphical representation of the workflow;
receiving, from the infusion device, user input to adjust or enter operational parameters according to the workflow;
adjusting the administration of the medication based on the user input; and
determining that the alert condition was resolved.

18. The non-transitory machine-readable storage medium of claim 15, wherein the operations further comprise:
graphical visualization comprises a copy of a user interface displayed on the infusion device and a replay of operational data modifications and patient physiological data as it appeared within the user interface between the start time and the end time, including the subset of the first physiological data and the subset of the plurality of modifications as it appeared within the user interface when received at the infusion device.

19. The non-transitory machine-readable storage medium of claim 15, wherein the operations further comprise:
assigning to the infusion session, by an infusion device, before an identity of the patient is known, a pseudo identifier representative of the patient;
receiving, by the infusion device, one or more modifications at least one operating parameter of the infusion device, the administration of the medication being adjusted according to the one or more modifications;
receiving, after the medication is adjusted, second physiological data associated with the patient and indicative of a response to the adjusted administration of the medication;
recording, in association with the pseudo identifier, the one or modifications and the physiological data to the infusion session;
receiving, after the medication is adjusted, a known patient identifier of a known patient for association with the administration of the medication; and
assigning the known patient identifier to the infusion session in substitution of the pseudo identifier;
generating a graphical representation of the infusion session, the graphical representation including a graphical visualization of the recorded one or more modifications and second physiological data in association with the known patient identifier, and at least one additional physiological data point received after the known patient identifier is assigned to the infusion session.

20. The non-transitory machine-readable storage medium of claim 15, wherein the operations further comprise:
assigning a pseudo identifier representative of an intravenous (IV) set to the infusion session, by an infusion device, before an identity of the IV set is known;
receiving, by the infusion device, one or more modifications at least one operating parameter of the infusion device, the administration of the medication being adjusted according to the one or more modifications;
receiving, after the medication is adjusted, second physiological data associated with the patient and indicative of a response to the adjusted administration of the medication;
recording, in association with the pseudo identifier, the one or modifications and the physiological data to the infusion session;
receiving, after the medication is adjusted, a known IV set identifier of a known type of IV set for association with the administration of the medication; and
assigning the known IV set identifier to the infusion session in substitution of the pseudo identifier;
generating a graphical representation of the infusion session, the graphical representation including a graphical visualization of the recorded one or more modifications and second physiological data in association with the known IV set identifier, and at least one additional physiological data point received after the known IV set identifier is assigned to the infusion session.
